(19) United States
(12) Patent Application Publication
(10) Pub. No.: US 2009/0164011 A1
(43) Pub. Date: Jun. 25, 2009

(54) Surface treatment of implants

(75) Inventors: Zongtao Zhang, Unionville, CT (US); Haitong Zeng, Oakland, NJ (US); Daniel E. Lawrynowicz, Cornwall, NY (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Howmedica Osteonics Corp., Mahwah, NJ (US)

(21) Appl. No.: 12/004,748

(22) Filed: Dec. 21, 2007

(51) Int. Cl. A61F 2/02

(52) U.S. Cl. 623/11.11

(57) ABSTRACT

An implant and a method for forming an implant. The implant includes a fixation surface having at least a portion made up of an oxidized material.

### CROSS-REFERENCE TO RELATED APPLICATIONS

This application is related to commonly owned concurrently filed U.S. patent application Ser. No. ______ (Attorney Docket No.: OSTEON 3.0-643) and commonly owned concurrently filed U.S. patent application Ser. No. ______ (Attorney Docket No.: OSTEON 3.0-685), the disclosures of which are hereby incorporated by reference herein.

### FIELD OF THE INVENTION

The present invention relates to surgical implants. More particularly, the invention relates to wear-resistant surgical implants.

### BACKGROUND OF THE INVENTION

The use of surgical implants to compensate for diseased, damaged, or missing anatomical elements is well known. In a common application, implants are used to replace skeletal elements, such as articulating bone joints and related bone structures. For example, total knee arthroplasty involves the replacement of portions of the patellar, femur and tibia with implants.

Some implants can last a few decades. However, a significant number fail within 10 to 15 years. While this may be acceptable for some older patients, longer service lives are needed as implants are used in increasing numbers of younger patients and more active older patients.

The service life of a surgical implant is largely dependent on the amount of wear and tear to which the implant's load bearing surface and fixation surface are exposed. Localized stress from interaction between each of these surfaces and respective opposing surfaces generates small particulate debris that breaks off and contaminates the tissues and fluid surrounding the implant. In response, the body's immune system will secrete enzymes in an attempt to degrade these particles. However, the enzymes often kill adjacent bone cells or cause osteolysis resulting in mechanical loosening and failure of the implant. Further, protuberances in the bearing surface can scratch an opposing surface, which leads to microcrack formation and ultimately to failure of the implant. Therefore, it is important to minimize the wear-rate of an implant's load bearing and fixation surfaces.

Accordingly, there is a need in the art for implants with improved surfaces. It would be particularly advantageous if such implants exhibited increased resistance to the release of wear debris, the formation of microcracks, and the expansion of microcracks. In this manner, the effective service of life of implants can be prolonged.

### SUMMARY OF THE INVENTION

In the interest of providing implants of improved durability, the present invention was conceived. The invention provides an implant and a method for forming an implant. The implant includes a fixation surface having at least a portion made up of an oxidized material.

### DESCRIPTION OF THE DRAWINGS

The following detailed description, given by way of example, but not intended to limit the invention solely to the specific embodiments described, may best be understood in conjunction with the accompanying drawings wherein like reference numerals denote like elements and parts, in which:

FIG. 1A is an anterior-posterior (AP) view of the skeletal structure of a lower left hand portion of the human body.

FIG. 1B is a lateral view of the portion shown in FIG. 1A.

FIG. 2 is an AP view of the skeletal portion of a left knee joint in flexion.

FIG. 3 is a perspective view of the knee joint of FIG. 2 as resected in preparation for attachment of femoral and tibial knee implants.

FIG. 4 is a perspective view of the knee joint of FIG. 3 in extension with an attached femoral implant, an attached tibial implant, an associated patella component, and an associated tibial insert.

FIG. 5 is an exploded view of a femoral implant, tibial implant, and tibial insert in accordance with an embodiment.

### DETAILED DESCRIPTION OF PREFERRED EMBODIMENTS

For purposes of brevity of description, the preferred embodiments will be described in the context of implants used in total knee arthroplasty. However, it should be noted that the embodiments are not limited to implants used in total knee arthroplasty. Upon review of this disclosure, one skilled in the art will readily appreciate how the described embodiments are applicable to a wide range of implants.

Further, for purposes of clarity of description, the following terminology is used. When referring to bones or other body parts, the term “proximal” means closest to the heart and the term “distal” means more distant from the heart.

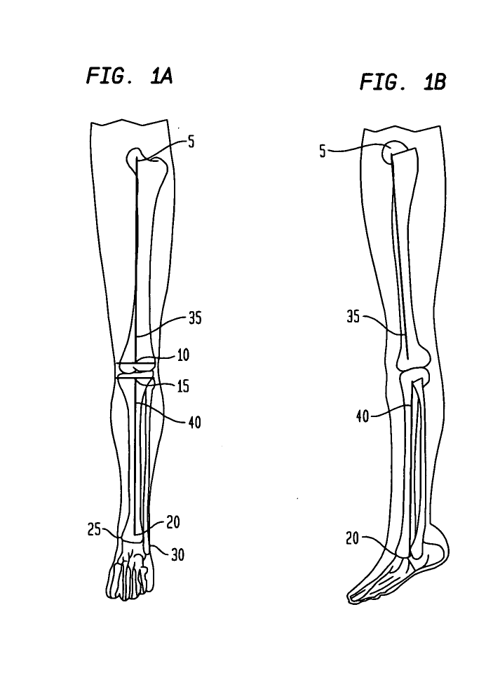

Referring now to FIG. 1A, there is shown an anterior-posterior (AP) view of the skeletal structure of a lower left hand portion of the human body. Several anatomical “landmarks” are noted. The landmarks include a center of the femoral head 5, a distal femur center 10, a tibia center 15, an ankle center 20, a medial malleolus 25, and a lateral malleolus 30. Further, a femoral axis 35 and a tibial axis 40 are defined. The femoral axis is defined by a line passing through the center of the femoral head and the center of the distal femur. The tibial axis is defined by a line passing through the tibia center and the ankle center.

FIG. 1B is a lateral view of the portion shown in FIG. 1A.

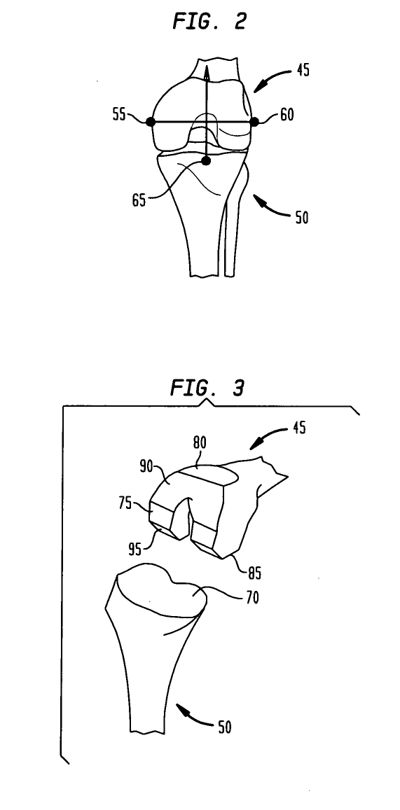

FIG. 2 is an AP view of the skeletal portion of a left knee joint in flexion. As can be seen from FIG. 2, the joint is formed where a distal femur portion 45 meets a proximal tibia portion 50. Anatomical landmarks shown in FIG. 2 include a medial epicondyle 55, a lateral epicondyle 60, and an anterior-posterior axis (or “Whiteside Line”) 65. In order to attach femoral and tibial knee implants (i.e. “articular bearing components of a resurfacing-type knee prostheses”) to the joint of FIG. 2, both the distal femur portion and the proximal tibia portion must be resected.

FIG. 3 is a perspective view of the knee joint of FIG. 2 as resected in preparation for attachment of femoral and tibial knee implants. As can be seen from FIG. 3, the tibia has been resected along a single plane 70, the proximal tibial resection plane. The femur has been resected along five resection planes, a distal femoral resection plane 75, an anterior femoral resection plane 80, a posterior femoral resection plane 85, a distal-anterior femoral resection plane 90, and a distal-posterior femoral resection plane 95. The tibial and femoral resection planes are oriented so as to mate with the tibial and femoral implants.

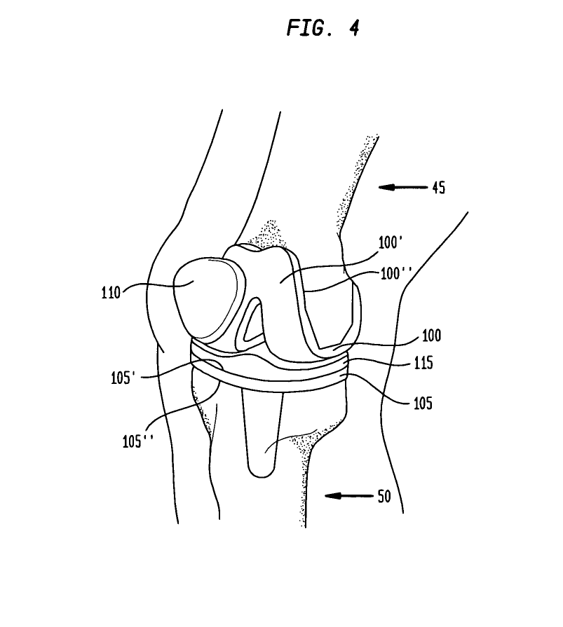

FIG. 4 is a perspective view of the knee joint of FIG. 3 in extension with an attached femoral implant 100, an attached tibial implant 105, an associated patella component 110, and an associated tibial insert 115. Typically, the femoral and tibial implants are made of metal, and the patella component and tibial insert are made of polyethylene. As can be seen from FIG. 4, the femoral implant is mated with the distal femur portion 45, and the tibial implant is mated with the proximal tibia portion 50. In particular, it can be seen how the resection planes (70-95 of FIG. 3) mate with surfaces of the femoral and tibial implants, how the femoral implant bears on the tibial insert, and how the tibial implant mates with the tibial insert.

The femoral and tibial implants shown in FIG. 4 are each said to include a “bearing surface” and a “fixation surface.” More particularly, the femoral implant is said to include a femoral implant bearing surface 100′ and a femoral implant fixation surface 100″, and the tibial implant is said to include a tibial implant bearing surface 105′ and a tibial implant fixation surface 105″.

It should be noted that tibial implant bearing surface 105′ is often referred to as the “tibial implant mating surface.” However, for purposes of facilitating the description and claims of this specification surface 105′ will be referred to as the tibial implant “bearing surface.”

Typically, the femoral implant and tibial implant are respectively attached to the distal femur and the proximal tibia through the use of acrylic bone cement. That is, the femoral implant is attached to the distal femur by using cement to adhere the femoral implant fixation surface to the resected surface of the distal femur, and the tibial implant is attached to the proximal tibia by using cement to adhere the tibial implant fixation surface to the resected surface of the proximal tibia. The cement is not shown in FIG. 4.

It has been recognized by the inventors that wear and corrosion limits the service life of implants like the one shown in FIG. 4. In particular, the inventors have recognized that wear debris and microcracks arise from (1) the motion at the interface between the femoral implant bearing surface and the tibial insert; (2) micro-motion at the interface between the femoral implant fixation surface and the cement that adheres the femoral implant to the distal femur; (3) micro-motion at the interface between the tibial implant fixation surface and the cement that adheres the tibial implant to the proximal tibia; and (4) micro-motion at the interface between the tibial implant bearing surface and the tibial insert. The normal articulation at the interface between the femoral implant bearing surface and tibial insert gives rise to articulation wear which generates a majority of the wear debris, while the micro-motion at the other interfaces gives rise to fretting wear and corrosion which generates a minority of the wear debris.

Attempts have been made to overcome the wear problem in knee implants. In particular, manufacturers have provided implants made of Ti-6Al-7Nb with a bearing surface hardened by oxygen diffusion and a fixation surface that is rough-peened or sandblasted (collectively “roughened”) for fixation without cement. However, there are several drawbacks associated with these types of implants. One drawback is that the oxygen diffused layer that makes up the bearing surface is a thin layer (approximately 20-50 um) of titanium oxide (TiO2), which has insufficient hardness (approximately 700-900 Vickers at 25 g load) to overcome the articulation wear problem, especially when the presence of bone chips precipitates the wear. Another drawback is that the roughened fixation surface does not allow for sufficiently durable fixation without the use of cement.

Further, manufactures have provided titanium-based implants having a fixation surface that is roughened for fixation with cement. However, such implants do not overcome the fretting wear and corrosion problems. The inventors have recognized that the fretting wear and corrosion problems may be related to the titanium's characteristics as a soft metal, and are therefore not overcome through roughening, either without cement or with cement.

In view of the drawbacks associated with prior implants, the inventors conceived and developed implants that make cement fixation workable.

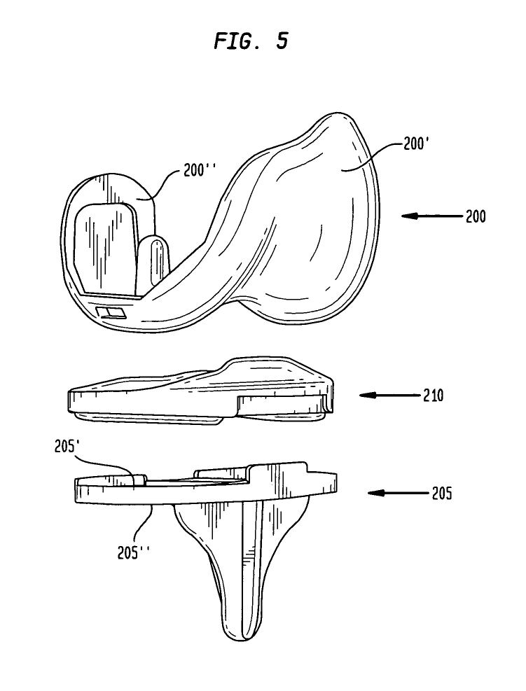

FIG. 5 shows an exploded view of a femoral implant 200, a tibial implant 205, and a tibial insert 210 in accordance with an embodiment. The femoral implant includes a bearing surface 200′ that is coated with chromium oxide (Cr2O3) and a fixation surface 200″ that is made up of an oxidized material. The tibial implant includes a bearing surface 205′ and a fixation surface 205″ that are made up of an oxidized material. In one implementation of the FIG. 5 embodiment, the base material of the femoral and tibial implants is titanium alloy. In such implementation, the bearing surface of the femoral implant is formed as a coating of chromium oxide over a titanium alloy base, and the oxidized material is oxidized titanium alloy. In another implementation, the base material of the femoral and tibial implants is titanium. In such other implementation, the bearing surface of the femoral implant is formed as a coating of chromium oxide over a titanium base, and the oxidized material is oxidized titanium. In either implementation the oxidized material may be TiO₂ or TiO₂₋ₓ, x<1. Further, in either implementation, the tibial insert may be made up of polyethylene.

It should be noted that each of the terms “coated” and “coating” as used in this disclosure include both cases in which the entire object of the term is coated and cases in which less than the entire object of the term is coated. Thus, when a bearing surface coated with chromium oxide is discussed in this disclosure, such surface may be entirely coated with chromium oxide or only partially coated with chromium oxide. Further, in cases of partial coating, such coating may be applied to a single unitary portion of the surface or to a multiple of unitary portions of the surface.

Similarly, it should be noted that each of the terms “oxidized” and “oxidizing” as used in this disclosure include both cases in which the entire object of the term is oxidized and cases in which less than the entire object of the term is oxidized. Thus, when an oxidized fixation surface is discussed in this disclosure, such surface may be entirely oxidized or only partially oxidized. Further, a partially oxidized fixation surface refers to either a surface in which only a single unitary portion of the surface is oxidized or a surface in which a multiple unitary portions of the surface are oxidized.

In any event, the femoral implant bearing surface of FIG. 5 may be generated by coating one or more areas of the femoral implant with Cr2O3 in accordance with the disclosures of commonly owned concurrently filed U.S. patent application Ser. No. ______ (Attorney Docket No.: OSTEON 3.0-643) and commonly owned concurrently filed U.S. patent application Ser. No. ______ (Attorney Docket No.: OSTEON 3.0-685), which are hereby incorporated by reference herein. The Cr2O3 coating may provide the implant with a bearing surface hardness greater than approximately 1000 Vickers at 300 g load. Accordingly, the surface hardness of the implants of FIG. 5 is substantially higher than the surface hardness of prior implants having TiO2 bearing surfaces (700-900 Vickers at 25 g load). The increased hardness decreases the wear-rate at the bearing-surface to tibial insert interface. Thereby, extending the lifespan of the implants.

The fixation surfaces of the FIG. 5 implants are harder than the fixation surfaces of prior titanium and titanium alloy implants. Also, the oxidized material of the fixation surfaces provides for surfaces of increased porosity relative to surfaces made of the same material in an un-oxidized state. The increased hardness and increased porosity of the fixation surfaces provide, in turn, for improved cement fixation of the implants and reduced wear-rates at the fixation surface interfaces. Accordingly, the oxidized fixation surfaces improve the longevity of the implants.

It should be noted that the femoral implant fixation surface and the tibial implant fixation surface of FIG. 5 are smooth surfaces. Alternatively, the femoral and/or tibial fixation surfaces may be roughened. As another alternative, the femoral and/or tibial fixation surfaces may be beaded.

As these and other variations and combinations of the features discussed above can be utilized without departing from the present invention as defined by the claims, the foregoing description of the preferred embodiments should be taken by way of illustration rather than by way of limitation of the invention as defined by the claims.

1. An implant, comprising:
a bearing surface having at least a portion made up of chromium oxide; and
a fixation surface having at least a portion made up of an oxidized material.

2. The implant as set forth in claim 1, wherein the oxidized material is titanium oxide.

3. The implant as set forth in claim 1, wherein the oxidized material is an oxidized alloy containing titanium.

4. The implant as set forth in claim 1, further comprising a base material of titanium.

5. The implant as set forth in claim 1, further comprising a base material of titanium alloy.

6. The implant as set forth in claim 1, wherein said at least a portion of the bearing surface has a hardness that is greater than approximately 1000 Vickers at 300 g load.

7. The implant as set forth in claim 1, wherein said oxidized material has an increased porosity relative to the material in an un-oxidized state.

8. An implant, comprising a fixation surface having at least a portion made up of an oxidized material.

9. The implant as set forth in claim 8, wherein the fixation surface is roughened.

10. The implant as set forth in claim 8, wherein the fixation surface is beaded.

11. A method for forming an implant, comprising the steps of:
coating at least a portion of a bearing surface of the implant with chromium oxide; and
oxidizing at least a portion of a fixation surface of the implant.

12. The method as set forth in claim 11, wherein the step of oxidizing comprises oxidizing at least a titanium portion of the fixation surface.

13. The method as set forth in claim 11, wherein the step of oxidizing comprises oxidizing at least a titanium alloy portion of the fixation surface.

14. The method as set forth in claim 11, further comprising a step of providing a base material of titanium prior to coating and oxidizing.

15. The method as set forth in claim 11, further comprising a step of providing a base material of titanium alloy prior to coating and oxidizing.

16. The method as set forth in claim 11, wherein, after coating, said at least a portion of the bearing surface has a hardness that is greater than approximately 1000 Vickers at 300 g load.

17. The method as set forth in claim 11, wherein, after oxidizing, said at least a portion of the fixation surface has an increased porosity relative to said at least a portion prior to oxidizing.

18. A method for forming an implant, comprising the step of oxidizing at least a portion of a fixation surface of the implant.

19. The method as set forth in claim 18, wherein the fixation surface is roughened.

20. The implant as set forth in claim 18, wherein the fixation surface is beaded.
